SEQUENCE LISTING (4 sequences)

SEQ ID NO: 1 - DNA, 480 bp, Homo sapiens
  misc_feature 8, 14, 24, 28, 170, 197, 390, 428, 431, 438, 460, 475: n = A,T,C or G
caactctncccctncctacggagncggntcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcgtcctntagcgatgataagctgtcaaacatganaattacaacttatatcgtatggggctgacttcaggtgctacatttgaagagataaattgcactgaaatctagcttgccaaacctacaggtggggtctttcaacaggataccactttttaattttattataatatggtaatgatactaacagacaaaattaggaaacagggaagaaggccaggtgcagtggctnacgcctgtaatcccagcactttgggagggcggggcggncanatcaccntgagttaggagctcaagaccancctggccaacatggngaaac

SEQ ID NO: 2 - DNA, 24 bp, Artificial Sequence
  primer
ctgttagtatcattaccatattat

SEQ ID NO: 3 - DNA, 24 bp, Artificial Sequence
  primer
tagaaaatgctaccatcaaatttg

SEQ ID NO: 4 - DNA, 26 bp, Artificial Sequence
  primer
cacctggtgcatgacccgcaagcccg

(19) United States
(12) Patent Application Publication
(10) Pub. No.: US 2005/0114913 A1
(43) Pub. Date: May 26, 2005

(54) Methods and compositions for generating homozygous mutations

(76) Inventor: Limin Li, Potomac, MD (US)

Correspondence Address: Supervisor Patent Prosccution Services;Piper Rudnick, 1200 Nineteenth Street NW, Washington, DC, 20036-2412, US

(21) Appl. No.: 10/490,829

(22) PCT Filed: Sep. 27, 2002
(86) PCT No.: PCT/US02/31017

(30) Foreign Application Priority Data
Sep. 27, 2001 (US) 60325497

(51) Int. Cl. A01K 67/027; C12N 5/06; C12N 15/85

(52) U.S. Cl. 800/18; 435/455; 435/354

(57) ABSTRACT

The invention provides methods and compositions for generating a homozygous mutation at genomic loci of polyploid cells, e.g., mammalian cells. The methods of the invention employ a gene search vectors comprising a selection marker linked to and under the control of a regulated promoter. A gene search vector is inserted into a genomic locus of a cell to produce a single allelic mutation. Double allelic mutation at the genomic locus is achieved by generating and selecting cells that have undergone homologous recombination that leads to homozygous insertion of the gene search vector, or portion thereof, at the genomic locus by culturing the polyploid cells under a concentration of an inducing agent to which the regulated promoter is responsive such that the activity level of the regulated promoter is tuned, e.g., reduced from full strength, to facilitate selection of cells containing homozygous mutation at the locus

This application claims the benefit under 35 U.S.C. § 119(e) of U.S. Provisional Patent Application No. 60/325,497, filed on Sep. 27, 2001, which is incorporated by reference herein in its entirety.

### FIELD OF THE INVENTION

The invention relates to methods and compositions for generating homozygous mutations at genomic loci in polyploid cells. The invention also relates to clones of cells and transgenic animals or plants which carry homozygous allelic mutations in one or multiple genes. The invention also relates to libraries of cells and libraries of transgenic animals or plants, comprising a plurality of different cells or transgenic animals or plants, each of which carries a different homozygous allelic mutation in one or multiple genes.

### BACKGROUND OF THE INVENTION

Understanding the biological function of mammalian genes remains one of the major challenges in the post genomic era. With the human genome sequenced, less than 20% of the estimated 30,000-50,000 genes (Venter et al, 2001 Science 291:5507; Lander, 2001, Nature 409:860) are well characterized with their biological function known. Gene inactivation techniques widely used for identifying and analyzing genes in bacteria and yeasts are not successful in higher eukaryotic systems, e.g., mammalian systems, due primarily to the diploid nature and complicity of an higher eukaryotic genome. For example, the identification of cells containing lesions that produce recessive phenotypes requires that multiple alleles of the gene be inactivated.

Many genetic approaches have been attempted for determination of gene function in mammalian cells, such as chemical mutagenesis, antisense oligonucleotides, retroviral insertions, ribozymes, gene targeting, and transgenic mice. However, each of these approaches has its limitations. Both chemical mutagenesis (Nolan et al, 2000, Nature Genetics 25:440-445) and retroviral insertions (Dougherty and Temin, 1991, Biotechnology 16:21-33) generate random mutations at single allelic sites in a mammalian genome. Since it is not practical to control in chemical mutagenesis the allelic site to be mutated, it is difficult to generate homozygous mutations and to identify the mutated genes. Methods based on antisense oligos (Wagner, 1994, Nature 372:333-335 ) and ribozymes (Hertel et al, 1996, EMBO J 15:3751) suffer the limitations of, e.g., particular selections of the target DNA sequences and low efficiency of oligo delivery. Gene targeting and transgenic mice (Capecchi, 1989, Science 244:1288-1292) are widely used, but are laborious and time consuming, and are therefore not suitable for gene function screening.

U.S. Pat. No. 6,139,833 discloses a method for gene discovery using a retrovirus that has been engineered to establish proviral integration at any location within the genome of the target cells. The engineered retroviruses exhibit increased accessibility to genomic DNA, and are useful for mutating and identifying the chromosomal target sequences of DNA binding proteins. The invention employs a combination of retroviral integrase/DNA binding protein fusion constructs and gene-trapping methodologies. The method allows for the generation of a collection of eukaryotic cells in which each cell contains a mutation in a target gene or sequence for a known DNA binding protein for rapid in vivo functional analysis.

U.S. Pat. No. 6,136,566 describes methods and vectors (both DNA and retroviral) for the construction of a library of mutated cells. The library contains mutations in essentially all genes present in the genome of the cells. The library is generated using vectors designed to replace the 3′ end of an animal cell transcript with a foreign exon or to insert foreign exons internal to animal cell transcripts to genetically altered cells that have been treated to stably incorporate one or more types of the vectors. The library and the vectors allow for methods of screening for mutations in specific genes, and for gathering nucleotide sequence data from each mutated gene to provide a database of tagged gene sequences.

U.S. Pat. No. 5,928,888 discloses a method of identifying portions of a genome, e.g. genomic polynucleotides, in a living cell using a polynucleotide encoding a protein with beta-lactamase (BL) activity that can be detected with a membrane permeant BL substrate. The method involves inserting a polynucleotide encoding a protein with BL activity into the genome of an organism, contacting the cell with a predetermined concentration of a modulator, and detecting BL activity in the cell, thereby identifying proteins or compounds that directly or indirectly modulate a genomic polynucleotide.

U.S. Pat. No. 6,025,192 discloses methods and compositions for improved mammalian complementation screening, functional inactivation of specific essential or non-essential mammalian genes, and identification of mammalian genes which are modulated in response to specific stimuli. In the invention, replication-deficient retroviral vectors containing a polycistronic message cassette, a proviral excision element, a proviral recovery element, a 5′ retroviral long terminal repeat (5′ LTR), a 3′ retroviral long terminal repeat (3′ LTR), a packaging signal, a bacterial origin of replication, and a bacterial selection marker are used to facilitate expression of cDNA or genomic DNA (gDNA) sequences in mammalian cells.

U.S. Pat. No. 6,069,010 discloses vectors and methods for increasing the efficiency of gene targeting procedures, and for generation of transgenic mice. The methods utilize a two vector system: a first vector comprises a sequence of interest from a mammalian genome and a second vector comprises a recombination sequence, which is similar or identical in sequence to a region of the first genomic sequence. The first and second vectors are chosen to have compatible origins of replication in a bacterial cell so that upon introduction of both vectors into a single bacterial cell, recombination occurs, inserting the second vector into the first vector at the recombination sequence to create a single knockout vector. The knockout vector is then introduced into embryonic stem (ES) cells at a random integration site in the genome of the ES cells to generate ES cells comprising a disrupted genomic sequence at one locus. The ES cells are then used to generate transgenic animals having a heterozygous or homozygous gene knockout.

U.S. Pat. Nos. 5,922,927 and 6,242,667 disclose transgenic animals carrying a transgene, integrated randomly or at a predetermined location within the genome of the animals, comprising a polynucleotide sequence encoding a fusion protein which activates transcription, the fusion protein comprising a first polypeptide which binds to a tet operator sequence in the presence of tetracycline or a tetracycline analogue operatively linked to a second polypeptide which activates transcription in eukaryotic cells, and methods for producing such tetracycline-regulated transgenic animals. Transcription of the tet operator-linked nucleotide sequence is stimulated by a transcriptional activator fusion protein composed of two polypeptides, a first polypeptide which binds to tet operator sequences in the presence of tetracycline operatively linked to a second polypeptide which activates transcription in eukaryotic cells.

U.S. Pat. No. 6,004,941 discloses a regulatory system which utilizes components of the tet repressor/operator/inducer system of prokaryotes to regulate gene expression in eukaryotic cells. This invention provides methods for using the regulatory system for regulating in a highly controlled manner the expression of a gene linked to one or more tet operator sequences. The methods involve introducing into a cell a nucleic acid molecule encoding a fusion protein which activates transcription, the fusion protein comprising a first polypeptide which binds to a tet operator sequence in the presence of tetracycline or a tetracycline analogue operatively linked to a second polypeptide which activates transcription in eukaryotic cells; and modulating the concentration of a tetracycline, or a tetracycline analogue, such that expression of the tet operator-linked gene in the cell is regulated.

A method permitting concurrent inactivation of multiple alleles of a gene at a random chromosomal locus in the genome of a mammalian cell has been described (see, e.g., U.S. Pat. Nos. 5,679,523; 5,807,995; 5,891,668; and 6,248,523; Li et al., 1996, Cell 85:319-329) for identifying and analyzing mammalian genes. The method employs a knockout construct which includes a positive selection marker sequence and, in a 5′ direction from the selection marker region sequence, a transcription initiation sequence responsive to a transactivation factor. The transcription initiation sequence is oriented for antisense RNA transcription in the direction away from the selection marker region sequence such that when activated by the transactivation factor, it initiates antisense RNA transcription extending from the knockout construct into the chromosomal locus flanking the knockout construct at its 5′ end. Thus, although only one allele of the gene is knocked out, antisense RNA transcripts inactivate the other allele or alleles. The inactivation of both gene copies leads to a change in cell phenotype which can be distinguished from the wild-type phenotype.

Homozygous mutation of the Smad4 (DPC4) gene was demonstrated by using two rounds of gene targeting (Zhou et al., 1998, Proc. Natl. Acad. Sci. U.S.A. 95:2412-2416). In this method, first allele deletion was achieved using a neo targeting vector. G418-resistant clones were screened by PCR and Southern blotting to obtain a clone that carries a homologous recombination and no additional random integrants. Second allele deletion at the same gene was then achieved by tranfecting the obtained clone with a Hygromycin targeting vector. Hygromycin-resistant clones were then screened by PCR and Southern blotting to obtain clones that carry homologous recombination at the Smad4 gene and no additional random integrants. Homozygous mutation in hSecurin gene was also demonstrated. In this method, in addition to two rounds of gene targeting, a step of excising the antibiotic resistance gene before the second round of gene targeting was employed (Jallepalli et al., 2001, Cell 105:445-457). Since the frequency of homologous recombination can be low, e.g., between 10⁻⁷ to 10⁻⁴ (see, e.g., U.S. Pat. Nos. 5,487,992; 5,627,059; 5,631,153; and 6,204,061), methods relying on two rounds of homologous recombination may not yield any clones with homozygous mutation. Therefore, such methods are not generally applicable for targeting any genomic locus. The yield problem is even more severe for generation of homozygous mutations at multiple genes. Further, the method requires extensive screening for targeted homologous recombination at both rounds of gene targeting, and is therefore extremely tedious and time consuming.

Accordingly, efficient methods for the generation of random or targeted homozygous mutations in one or more genes to produce cells having a particular phenotype are highly desirable. There is therefore a need for methods which permit not only more reliable generation of homozygous mutations in cells but also more efficient screening procedures. There is also a need for methods for generating libraries of cells, e.g., mammalian embryonic stem (ES) cells, comprising different mutated cells, each of which carries a different mutated gene or genes. There is also a need for methods for generating transgenic animals or plants, and libraries thereof, which carry mutated gene or genes.

Discussion or citation of a reference herein shall not be construed as an admission that such reference is prior art to the present invention.

### SUMMARY OF THE INVENTION

The invention provides methods and compositions for generating a homozygous mutation at a genomic locus of polyploid cells, e.g., mammalian cells. The method of the invention employs a gene search vector comprising a selection marker gene linked to and under the control of a regulated promoter. The gene search vector is inserted into a genomic locus of a cell to produce a single allelic mutation. Double allelic mutation at the genomic locus is achieved by generating and selecting cells that have undergone homologous recombination that leads to homozygous insertion of the gene search vector, or portion thereof, at the genomic locus by a method comprising adjusting the concentration, and/or applying analogues, of the inducing agent under a given selection pressure such that the activity level of the regulated promoter is tuned, e.g., reduced from full strength, to facilitate selection of cells containing homozygous mutation at the locus.

The invention provides DNA constructs that can be used for generating a homozygous mutation at a genomic locus in a type of polyploid cells. The DNA constructs of the invention comprise a selection marker gene, a regulated promoter, an optional reporter gene, and an optional rapid cloning element. The selection marker in the gene search vector can be any selection marker known in the art for a particular type of cells, e.g., a particular type of mammalian cells. Preferably, the selection marker is a selection marker that confers distinct characteristics to cells that carry the gene encoding the selection marker at both alleles of a genomic locus such that these cells can be identified and/or separated from cells that carry the gene at only one allele of a genomic locus. The distinct characteristics can be a cell's ability to resist to a drug. For example, a gene dosage dependent drug resistance. Or, alternatively, can be a characteristic that allows differential detection to identify cells carrying double insertion from cells carrying single insertion. Preferably, the level of activity of the selection marker can be controlled such that cells can be selected under a particular level of selection, i.e., a particular level of selection pressure. More preferably, the range of quantification can be further enhanced by the regulated promoter. In one embodiment, a drug resistance gene is used as the selection marker. Drug resistance genes that can be used in the present invention include, but are not limited to, Neomycin/G418, Puromycin, Hygromycin B, Zeocin, or mycophenolic acid resistance genes. In another embodiment, a cell surface marker is used as the selection marker. Cell surface marker genes that can be used in the present invention include, but are not limited to, genes encoding CD4, CD8, CD20, HA, or any synthetic or foreign cell surface markers. In still another embodiment, a fluorescence marker is used as the selection marker. Fluorescent markers that can be used in the present invention include, but are not limited to, genes encoding green fluorescence protein (GFP), blue fluorescence protein (BFP), red fluorescence protein (RFP), or any variants thereof.

The regulated promoter in the gene search vector can be any transcription regulation system known in the art for the chosen type of cells. Preferably, the regulated promoter is highly inducible in a dosage and/or analogue dependent manner. In one embodiment, a tetracycline regulated gene expression system is used. In another embodiment, an ecdyson regulated gene expression system is used. In still another embodiment, a MMTV glucocorticoid response element regulated gene expression system is used.

The reporter gene in the gene search vector can be any gene known in the art that encodes a measurable and selectable marker in the chosen type of cells, e.g., a type of mammalian cells. In one embodiment, the reporter gene is a gene encoding β-galactosidase. In another embodiment, the reporter gene is a gene encoding β-geo. In still another embodiment, the reporter gene is a drug resistance gene, such as but not limited to Neomycin/G418, Puromycin, Hygromycin B, Zeocin, or mycophenolic acid resistance gene. In still another embodiment, the reporter gene is a gene encoding a cell surface marker, such as but not limited to a gene encoding CD4, CD8, CD20, HA, or any synthetic or foreign cell surface marker. The reporter gene can also be a gene encoding a fluorescent marker, such as but not limited to a gene encoding green fluorescence protein (GFP), blue fluorescence protein (BFP), red fluorescence protein (RFP), or any variants thereof. Preferably, the reporter gene encodes a different marker from the selection marker. In a preferred embodiment, the reporter gene is oriented in the opposite orientation as the regulated promoter and is located either upstream or downstream of the regulated promoter. In another embodiment, the reporter gene is oriented in the same orientation as the regulated promoter and is located upstream of the regulated promoter to avoid false report activity.

The optional rapid cloning element in the gene search vector of the invention comprises a bacterial plasmid replication origin and a bacterial selection marker. Any bacterial plasmid replication origin, such as but not limited to Ori, colEI, pSC101, pUC, or f1 phage ori, can be used. Any bacterial selection markers, such as but not limited to, chloramphenicol, ampicillin, tetracycline, or kanamycin, can be used in the present invention.

The invention provides a method for generating a plurality of polyploid cells having a homozygous mutation at a genomic locus from one or more polyploid cells comprising a DNA construct integrated at one allele of said genomic locus. The DNA construct comprises a selection marker gene linked to a regulated promoter. The method comprises culturing the polyploid cells under a concentration of an inducing agent to which the regulated promoter is responsive under a given selection condition such that polyploid cells having the homozygous mutation constitute at least a predetermined percentage of the resultant cell population within a given period of time. The method therefore allows retrieving cells having the homozygous mutation. In a preferred embodiment, the concentration of the inducing agent or an analogue of the inducing agent is set such that homozygous mutation at the locus resulting in an 100% increase of selection marker activity. In another preferred embodiment, the concentration of the inducing agent or an analogue of the inducing agent is chosen such that under a given selection condition and within a given period of time cells having homozygous mutation constitute at least a predetermined percentage of the resultant population of cells. Preferably, the concentration of the inducing agent or an analogue of the inducing agent is chosen such that under a given selection condition cells having homozygous mutation constitute at least 1%, 5%, 10%, 20%, 50%, or 90% of the resultant population of cells within a given period of time. Preferably, the given period time is 24 hours, 72 hours, 7 days, 14 days, or 28 days.

In one embodiment, cells comprising a DNA construct of the invention integrated at one allele of the genomic locus are generated by random insertion of the DNA construct in the genomes of cells.

In another embodiment, cells comprising a DNA construct integrated at one allele of the genomic locus are generated by targeted insertion of the DNA construct in a chosen genomic locus of cells. Preferably, the single allelic mutation at the chosen genomic locus is generated by homologous recombination. In one embodiment, a positive-negative selection scheme for targeted insertion by homologous recombination that uses a negative selection marker is employed. In another embodiment, a method utilizing a cell surface marker for selection against random integrations is employed. In this embodiment, selection for the absence of the cell surface marker is carried out by contacting the transfected cells with a binding molecule to identify and remove cells expressing the cell surface marker.

In a preferred embodiment, a method utilizes a selection scheme in which a selection marker gene that encodes a fluorescence protein, such as a green fluorescence protein, is employed for selection against random, non-homologous, insertions. The method utilizes a gene targeting vector comprising a first sequence region comprising a nucleotide sequence which is substantially homologous to a first DNA sequence in the chosen genomic locus; a second sequence region comprising a nucleotide sequence which is substantially homologous to a second DNA sequence in the chosen genomic locus; a third sequence region positioned between the first and second DNA sequence regions and comprising the DNA construct of the invention to be inserted, i.e., a DNA construct comprising a selection marker gene, a regulated promoter, a reporter gene for reporting integration of the gene search vector in the genome of the targeted cells, and an optional rapid cloning element; and a fourth sequence region comprising a nucleotide sequence located at 5′ to the first or 3′ to the second sequence region encoding a fluorescence marker for selection against random integration. Transfected cells that carry the insertion of the first through third sequence regions in the genome by homologous recombination can be selected by selecting for the presence of the positive selection marker activity and the absence of fluorescence from the fluorescence marker encoded in the outside region, i.e., the fourth sequence region. (See, e.g., Limin Li, U.S. Provisional Patent Application No. 60/325,450, filed on Sep. 27, 2001, which is incorporated herein by reference in its entirety.)

In a preferred embodiment of the method that utilizes a fluorescence marker for selection against random, non-homologous, insertions, a drug resistance gene is used as the reporter gene for reporting integration of the DNA construct in the genome of the targeted cells. In another preferred embodiment, a fluorescence marker is used as the reporter gene for reporting integration of the DNA construct in the genome of the targeted cells. In this embodiment, the selection for cells carrying the insertion of the gene search vector is preferably achieved by FACS. In both embodiments, the selection against random, non-homologous, integration of the gene search vector can be carried out by detecting the fluorescence from the fluorescence marker encoded in the fourth sequence region using any fluorescence based cell sorting methods known in the art, e.g., by FACS. The method allows production of a cell population in which cells that carry the single allelic insertion of the DNA sequence by homologous recombination constitute at least 10%, 30%, 50%, 70%, or 90% of the population.

In a specific embodiment, the invention provides a method for generating homozygous mutations at a genomic locus in a type of polyploid cells, comprising (a) integrating a DNA construct comprising a selection marker gene linked to a regulated promoter at one allele of said genomic locus in one or more cells of said type of polyploid cells; and (b) culturing said one or more cells under a concentration of an inducing agent to which said regulated promoter is responsive under a given selection condition such that cells having said homozygous mutation at said genomic locus constitute at least a predetermined percentage of the resultant cell population within a given period of time. In a preferred embodiment, the concentration of the inducing agent or an analogue of the inducing agent is set such that homozygous mutation at the locus resulting in an 100% increase of selection marker activity. In another preferred embodiment, the concentration of the inducing agent or an analogue of the inducing agent is chosen such that under a given selection condition and within a given period of time cells having homozygous mutation constitute at least a predetermined percentage of the resulted population of cells. Preferably, the concentration of the inducing agent or an analogue of the inducing agent is chosen such that under a given selection condition cells having homozygous mutation constitute at least 1%, 5%, 10%, 20%, 50%, or 90% of the resulted population of cells within a given period of time. Preferably, the given period time is 24 hours, 72 hours, 7 days, 14 days, or 28 days.

The invention also provides methods for generating homozygous mutations at a plurality of genomic loci in a type of polyploid cells. The methods can be used to generate, from one or more polyploid cells comprising a plurality of DNA constructs, each DNA construct comprising a selection marker linked to one of a plurality of regulated promoters and integrated at one allele of one of the plurality of genomic loci, a plurality of polyploid cells having homozygous mutations at the plurality of genomic loci by culturing said one or more polyploid cells under a concentrations of each of a plurality of inducing agents to which one of said regulated promoters is responsive under a given selection condition such that said plurality of polyploid cells having said plurality of homozygous mutations constitute at least a predetermined percentage of the resultant cell population within a given period of time. The method therefore allows retrieving cells having the homozygous mutation. In a specific embodiment, the invention provides a method for generating homozygous mutations at a plurality of genomic loci in a type of polyploid cells, comprising (a) integrating a plurality of DNA constructs each at one allele of one of said plurality of genomic loci in one or more polyploid cells of said type of polyploid cells, wherein each of said plurality of DNA construct comprises a selection marker gene linked to a regulated promoter; and (b) culturing said one or more polyploid cells under a concentration of each of a plurality of inducing agents to which one of said regulated promoters is responsive under a given selection condition such that said plurality of polyploid cells having said plurality of homozygous mutations constitute at least a predetermined percentage of the resultant cell population within a given period of time. In a preferred embodiment, the concentrations of the inducing agents or analogues of the inducing agents are set such that homozygous mutations at the loci resulting in an 100% increase of selection markers' activities. In another preferred embodiment, the concentrations of the inducing agents or analogues of the inducing agents are chosen such that under given selection conditions and within a given period of time cells having homozygous mutations constitute at least a predetermined percentage of the resulted population of cells. Preferably, the concentration of the inducing agent or an analogue of the inducing agent is chosen such that under a given selection condition cells having homozygous mutation constitute at least 1%, 5%, 10%, 20%, 50%, or 90% of the resulted population of cells within a given period of time. Preferably, the given period time is 24 hours, 72 hours, 7 days, 14 days, or 28 days.

The invention also provides a population of polyploid cells, comprising cells that carry a homozygous insertion of a DNA construct at a genomic locus, wherein the DNA construct comprises a selection marker linked to a regulated promoter. The population of polyploid cells comprising cells that carry homozygous insertion can be any cells, such as but not limited to any type of cells from any animal or any plant.

The invention also provides a library of polyploid cells, comprising a plurality of different polyploid cells, each of said plurality of different polyploid cells carries different homozygous mutations in one or more genes. Preferably, the library of cells of the invention consists of at least 10, 100, 1,000, 10,000 different cells, each carrying different homozygous mutations in one or more genes. More preferably, the library of cells of the invention comprises for each gene in the genome of the type of cells at least one cell which carries a homozygous mutation in the gene.

The invention further provides a method for generating a transgenic animal, said transgenic animal carrying a homozygous insertion of a DNA construct at a genomic locus, said DNA construct comprising a selection marker linked to a regulated promoter, said method comprising (a) integrating said DNA construct at one allele of said genomic locus in one or more embryonic stem cells of said animal; (b) culturing said one or more embryonic stem cells under a concentration of an inducing agent to which said regulated promoter is responsive under a given selection condition such that embryonic stem cells having said homozygous mutation constitute at least a predetermined percentage of the resultant embryonic stem cell population within a given period of time; and (c) retrieving said embryonic stem cells having said homozygous mutation; and (d) generating said transgenic animal using said retrieved embryonic stem cells. In a preferred embodiment, the concentration of the inducing agent or an analogue of the inducing agent is set such that homozygous mutation at the locus resulting in an 100% increase of selection marker activity. In another preferred embodiment, the concentration of the inducing agent or an analogue of the inducing agent is chosen such that under a given selection condition and within a given period of time the ES cells having homozygous mutation constitute at least a predetermined percentage of the resulted population of ES cells. Preferably, the concentration of the inducing agent or an analogue of the inducing agent is chosen such that under a given selection condition ES cells having homozygous mutation constitute at least 1%, 5%, 10%, 20%, 50%, or 90% of the resulted population of ES cells within a given period of time. Preferably, the given period time is 24 hours, 72 hours, 7 days, 14 days, or 28 days.

The invention further provides a transgenic organism, said transgenic organism carries homozygous insertion of a DNA construct at a genomic locus, wherein said DNA construct comprises a selection marker linked to a regulated promoter. The transgenic organism of the invention can be a transgenic animal, e.g., a trangenic mouse, or a transgenic plant. The invention further provides a library of transgenic organisms, comprising a plurality of different transgenic organisms of a same organism, each of said different transgenic organisms carries different homozygous mutations in one or more genes. Preferably, the library of transgenic organisms of the invention consists of at least 10, 100, 1,000, 10,000 different transgenic organisms, each carrying different homozygous mutations in one or more genes. More preferably, the library of transgenic organisms of the invention comprises for each gene in the genome of the transgenic organisms at least one transgenic organism which carries a homozygous mutation in the gene. In one embodiment, the library of transgenic organisms is a library of transgenic mice.

### BRIEF DESCRIPTION OF FIGURES

FIG. 1 shows a schematic illustration of gene search vectors of the invention.

FIGS. 2A and 2B show two embodiments of integration of the gene search vector of the invention into a genomic locus. FIG. 2A: the gene search vector integrated behind a chromosomal promoter; FIG. 2B: the gene search vector integrated upstream of a chromosomal promoter.

FIG. 3 is a schematic illustration of a method for generating double insertion by adjusting the concentration of the inducing agent to which the regulated promoter is responsive.

FIG. 4 shows a schematic illustration of conversion of single allelic insertion to double allelic insertion when the gene search vector is integrated behind the chromosomal promoter.

FIG. 5 shows a schematic illustration of conversion of single allelic insertion to double allelic insertion when the gene search vector is integrated upstream of the chromosomal promoter.

FIG. 6 shows a schematic illustration of cloning of genomic sequences by restriction digestion.

FIG. 7 shows a schematic illustration of generation of libraries of cells and transgenic animals using the method for generating homozygous mutations.

FIG. 8 shows a genomic DNA sequence adjacent to the insertion site of the gene search vector in the human UDP-glucose dehydrogenase gene.

FIG. 9 illustrates the location of the insertion site in the genomic locus of the human UDP-glucose dehydrogenase gene.

FIG. 10 shows results of genomic PCR analysis of cell lines containing mutations in the human UDP-glucose dehydrogenase gene. Three PCR primers were designed to identify the wild-type allele and the mutated allele. P1 and P2 PCR product (350 bp) identifies the wild-type allele, whereas P1 and P3 identifies the mutated allele. Clone 4 (lane 4) contains only mutated alleles.

### DETAILED DESCRIPTION OF THE INVENTION

The invention provides methods and compositions for generating homozygous mutations at genomic loci of polyploid cells, e.g., mammalian cells. The methods and compositions of the invention are useful for generating mutations at both alleles of a gene or genes in cells of any organism that has a polyploid genome. For example, the methods and compositions of the invention can be used to generate homozygous mutations in cells of a mammal or a plant. Any cell type or tissue can be used in the present invention.

The methods of the invention employ a gene search vector comprising a selection marker. The selection marker can be a selection marker that confers distinct characteristics to cells that carry the gene encoding the selection marker in both alleles of a genomic locus such that these cells can be identified and/or separated from cells that carry the gene in only one allele of the genomic locus. The gene search vector is first inserted into a genomic locus of a cell to produce a single allelic mutation. Double allelic mutation at the genomic locus, or double conversion, is achieved by selecting cells that carry two copies of the selection marker gene.

The selection marker can be linked to and under the control of a regulated promoter. The gene search vector is inserted into a genomic locus of a cell to produce a single allelic mutation. Double allelic mutation at the genomic locus, or double conversion, is achieved by generating and selecting cells carrying insertions at both alleles of the genomic locus by a method comprising adjusting the concentration, and/or applying analogues, of the inducing agent such that the activity level of the regulated promoter is tuned, e.g., reduced from fill strength, to facilitate selection of cells containing homozygous mutation at the locus under a given selection pressure. Alternatively, the selection pressure can be increased whereas the activity of the regulated promoter remains the same to achieve the same result. The invention is based, at least in part, on the discovery that particular activity level of a regulated promoter results in a particular level of transcription of the selection marker, which, under a chosen selection pressure, encourages insertion of the gene vector into the other allele of the genomic locus by homologous DNA recombination. For example, reduction of the activity level of the regulated promoter decreases the level of transcription of the selection marker; thus, under the same selection pressure, only cells which have undergone conversion of heterozygous genomic mutation to homozygous genomic mutation by homologous DNA recombination at the same locus have an adequate level of transcription of the selection marker to survive the selection pressure.

The methods of the invention permit generation of clones of cells, including but not limited to cells of any type or from any tissue of an animal, e.g., a mammal, or a plant, which carry homozygous allelic mutations in one or multiple genes. The invention also provides libraries of mutated cells, e.g., mutated animal or plant cells, comprising a plurality of different cells, each of which carries different homozygous allelic mutations in one or multiple genes. In a particularly useful embodiment, the methods of the invention are used in generation of embryonic stem cells carrying homozygous allelic mutations in one or multiple genes. The invention also provides methods for generating, from embryonic stem cells, transgenic animals which carry the mutated gene or genes as well as libraries of transgenic animals, each carries different homozygous mutations in one or more genes.

As used herein, a “homozygous mutation” refers to a mutation at two or more alleles of a gene in a cell having a polyploid genome, e.g., at both alleles of a gene in a cell having a diploid genome.

As used herein, a “library of cells” refers to a collection of cells comprising a plurality of different cells, each of which carries homozygous insertions of one or more gene search vectors of the present invention at one or more genomic loci. Preferably, the library of cells of the invention consists of at least 10, 100, 1,000, 10,000 different cells, each carrying different homozygous mutations in one or more genes. More preferably, the library of cells of the invention comprises for each gene in the genome of the cell at least one cell which carries a homozygous mutation in the gene.

As used herein, an “animal” refers to any higher eukaryotic animal that has a polyploid genome. An animal can be, but is not limited to, a mammal, e.g. a mouse. As used herein, a “stem cell” refers to an undifferentiated cell that can divide virtually indefinitely.

As used herein, a “plant” refers to any plant organism that has a polyploid genome. Plants that can be used in the present invention include, but not limited to, crop plants, e.g., wheat and rice, etc.

Gene Search Vectors and Methods of Introduction

The invention provides gene search vectors comprising a selection marker linked to a regulated promoter. As used herein, a “gene search vector” refers to a vector that can be used to insert a DNA construct contained in the vector into a genomic locus and to inactivate or activate the allele of the gene.

As used herein, a “selection marker” refers to a nucleotide sequence encoding a product that can be used in the selection and identification of the cells carrying the gene. As used herein, a “cell surface marker” refers to any cell surface marker that can be recognized by a binding molecule, e.g., an antibody.

As used herein, a “regulated promoter” refers to a promoter that can be activated when an appropriate inducing agent is present. As used herein, an “inducing agent” can be any molecule that can be used to activate transcription by activating a regulated promoter. An inducing agent can be, but is not limited to, a peptide or polypeptide, a hormone, or an organic small molecule. An “analogue” of an inducing agent is also often used. An analogue of an inducing agent refers to any molecule that activates the regulated promoter as the inducing agent does. However, the level of activity of the regulated promoter induced by different analogues may be different.

As used herein, a “rapid cloning element” refers to a nucleotide sequence which can be used to facilitate the cloning of the genomic sequences flanking the integration site in a host, e.g., in a bacterial host. In the present invention, a rapid cloning element comprising a replication origin is often used. As used herein, an “origin” or “replication origin” refers to a bacterial replication origin sequence. Preferably, the replication origin sequence comprises all necessary sequences for initiation of replication and segregation.

Gene Search Vectors

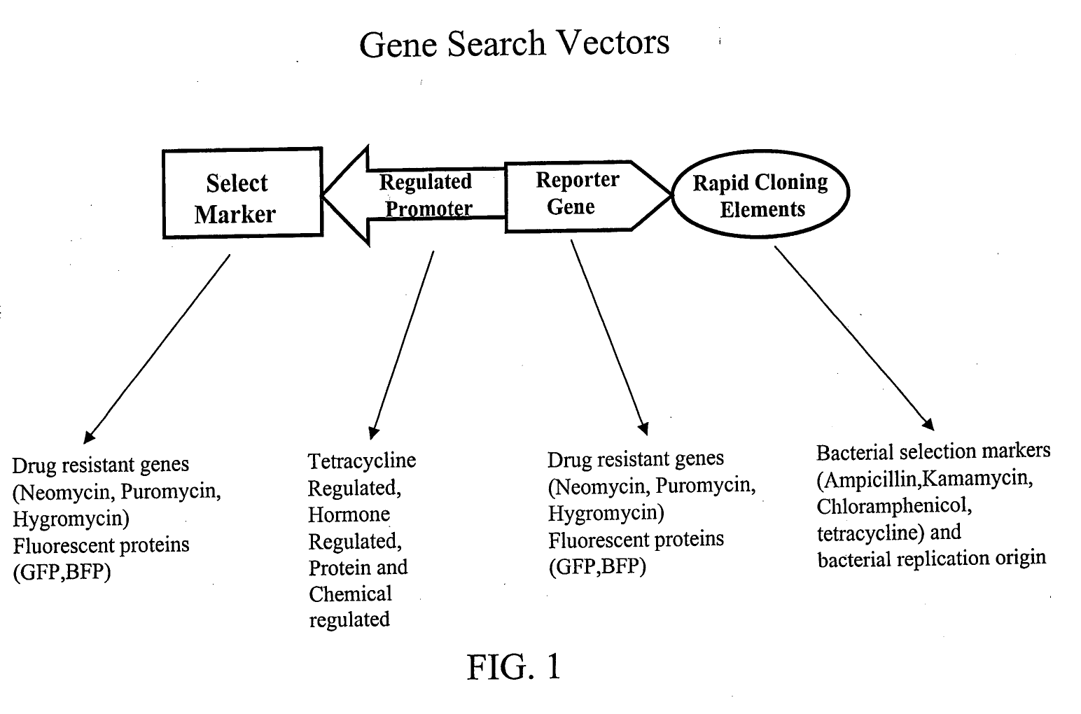

The invention provides methods of generating random and targeted homozygous mutations in polyploid cells, e.g., mammalian cells, using a gene search vector which comprises a DNA construct comprising four components: a selection marker gene, an optional promoter, e.g., a regulated promoter, an optional reporter gene, and an optional rapid cloning element, see a schematic illustration in FIG. 1. The gene search vector is constructed in such a manner that the DNA construct comprising the four components is integrated in the genome of the target cells.

Selection Marker Gene

The selection marker gene in the gene search vector can be any selection marker gene known in the art for a particular type of cells, e.g., a particular type of mammalian cells. Preferably, the selection marker is a selection marker that confers distinct characteristics to cells that carry the gene encoding the selection marker in both alleles of a genomic locus such that these cells can be identified and/or separated from cells that carry the gene in only one allele of the genomic locus. A distinct characteristic can be resistance to a drug. Or, alternatively, it can be a characteristic that allows differential detection for identification of cells carrying double insertion from cells carrying single insertion. In one embodiment, a drug resistance gene is used as the selection marker. Drug resistance genes that can be used in the present invention include, but are not limited to, Neomycin/G418, Puromycin, Hygromycin B, Zeocin, or mycophenolic acid resistance genes. In another embodiment, a cell surface marker is used as the selection marker. Cell surface marker genes that can be used in the present invention include, but are not limited to, genes encoding CD4, CD8, CD20, HA, or any synthetic or foreign cell surface markers. In still another embodiment, a fluorescence marker is used as the selection marker. Fluorescent markers that can be used in the present invention include, but are not limited to, genes encoding green fluorescence protein (GFP), blue fluorescence protein (BFP), red fluorescence protein(RFP), or any other modified fluorescent protein markers (see, e.g., Autofluorescent Proteins available at http://www.qbiogene.com/protocols/gene-expression/m-afp.pdf (accessed Sep. 5, 2001); Ellenberg et al., 1999, Trends in Cell Biol 9:52-56; Mizuno et al., 2001, Biochem. 40:2502-10; and Living Colors® User Manual, published Aug. 30, 2000, available at http://www.clontech.com/techinfo/manuals/PDF/PT2040-1.pdf (accessed Sept. 5, 2001)). Other genes can also be used as the selection marker gene in the present invention. For example, an oncogen gene, such as but not limited to Ras (see, e.g., Hahn et al., 1999, Nature 400:464-8), Myc (see, e.g., Littlewood et al., 1990, Adv. Dent. Res. 4:69-79), p53 mutants (see, e.g., Vogelstein et al., 1992, Cell 70:523-6), MDM2 (see, e.g., Chen et al., 1996, Mol. Cell. Biol. 16:2445-52), can be used as the selection marker. A cell cycle regulating gene, such as but not limited to Cyclin D1 (see, e.g., Motokura et al., Curr. Opin. Genet. Dev. 3:5-10), can also be used as the selection marker gene in the present invention.

Preferably, the selection marker is a selection marker that confers a distinct characteristic to cells that carry the gene encoding the selection marker at both alleles of a genomic locus such that these cells can be identified and/or separated from cells that carry the gene at only one allele of a genomic locus. The distinct characteristics can be a cell's ability to resist to a drug, for example, a gene dosage dependent drug resistance. Or, alternatively, it can be a characteristics that allows differential detection to identify cells carrying a double insertion from cells carrying a single insertion. Preferably, the level of activity of the selection marker can be controlled such that cells can be selected under particular level of selection. More preferably, the range of quantification can be further enhanced by the regulated promoter. In a preferred embodiment, a drug resistance gene is used as the selection marker. In this embodiment, level of selection is determined by the concentration of the drug, and cells can be selected in different concentrations of the drug. Any desired level of selection, i.e., any particular concentration of the drug, e.g., a level of selection that is optimized for selecting cells carrying homozygous mutations, can be determined by any methods known in the art. In some embodiments, the level of selection depends on the level of activity of the regulated promoter. In such embodiments, the desired selection level can be determined after or at the same time the level of activity of the regulated promoter is determined. In another preferred embodiment, a fluorescence marker is used as the selection marker. In this embodiment, level of selection is determined by the fluorescence intensity using any one of the known fluorescence activated cell sorter (FACS) methods. For example, the level of selection can be set to select cells having fluorescence intensities produced by cells carrying two copies of the fluorescence markers.

It will be apparent to one skilled in the art that any selection marker genes that are functionally equivalent to any of the selection marker genes as described herein, including any genes that are modified or mutated from any of the described selection marker genes, are also within the scope of the present invention.

Regulated Promoter Gene

The gene search vector of the present invention can comprise an optional regulated promoter. The regulated promoter in the gene search vector can be any mammalian transcription regulation system known in the art (see, e.g., Gossen et al, 1995, Science 268:1766-1769; Lucas et al, 1992, Annu. Rev. Biochem. 61:1131; Li et al., 1996, Cell 85:319-329; Saez et al., 2000, Proc. Natl. Acad. Sci. USA 97:14512-14517; and Pollock et al., 2000, Proc. Natl. Acad. Sci. USA 97:13221-13226). In one embodiment, a tetracycline regulated gene expression system is used (see, e.g., Gossen et al, 1995, Science 268:1766-1769). In another embodiment, an ecdyson regulated gene expression system is used (see, e.g., Saez et al., 2000, Proc. Natl. Acad. Sci. USA 97:14512-14517). In still another embodiment, a MMTV glucocorticoid response element regulated gene expression system is used (see, e.g., Lucas et al, 1992, Annu. Rev. Biochem. 61:1131). Other protein or chemical regulated gene expression systems can also be used (see, e.g., Li et al., 1996, Cell 85:319-329).

Preferably, the regulated promoter is highly inducible in a dosage and/or analogue dependent manner. In one embodiment, the level of activity of the regulated promoter is tuned to a desired level by a method comprising adjusting the concentration of the inducing agent to which the regulated promoter is responsive. Any desired level of activity of the regulated promoter, i.e., any particular concentration of the inducing agent, e.g., a level of activity that is optimized for generating and selecting cells carrying homozygous mutations, can be determined by any methods known in the art. In some embodiments, the level of activity depends on the level of selection of the selection marker. In such embodiments, the desired level of activity of the regulated promoter can be determined after or at the same time the level of selection of the regulated promoter is determined. More preferably, the regulated promoter is highly inducible with minimal background.

It will be apparent to one skilled in the art that any transcription regulation systems that are functionally equivalent to any of the systems as described, including any systems that are modified or mutated from any of the described systems, are also within the scope of the present invention.

Reporter Gene

The gene search vector of the present invention can comprise an optional reporter gene. The reporter gene can be any gene known in the art that encodes a measurable and selectable marker in the type of cells, e.g., a type of mammalian cells. In one embodiment, the reporter gene is a gene encoding β-galactosidase. In another embodiment, the reporter gene is a gene encoding β-geo. In still another embodiment, the reporter gene is a drug resistance gene, such as but not limited to Neomycin/G418, Puromycin, Hygromycin B, Zeocin, or mycophenolic acid resistance gene. In still another embodiment, the reporter gene is a gene encoding a cell surface marker, such as but not limited to a gene encoding CD4, CD8, CD20, HA, or any synthetic or foreign cell surface marker. The reporter gene can also be a gene encoding a fluorescent marker, such as but not limited to a gene encoding green fluorescence protein (GFP), blue fluorescence protein (BFP), red fluorescence protein(RFP), or any variants thereof (see, e.g., Autofluorescent Proteins available at http://www.qbiogene.com/protocols/gene-expression/m-afp.pdf (accessed Sept. 5, 2001); Ellenberg et al., 1999, Trends in Cell Biol 9:52-56; Mizuno et al., 2001, Biochem. 40:2502-10; and Living Colorsg User Manual, published Aug. 30, 2000, available at http://www.clontech.com/techinfo/manuals/PDF/PT2040-1.pdf (accessed Sept. 5, 2001)).

Preferably, the reporter gene is a different marker from the selection marker. Preferably, the reporter gene comprises a splicing acceptor at its 5′ end that allows fusion of the reporter gene to the RNA transcript from the upstream exons (see, e.g., Li et al., 1996, Cell 85:319-329). The reporter gene can be placed in either orientation in relation to other components in the gene search vector. In a preferred embodiment, the reporter gene is oriented in the opposite orientation as the regulated promoter. In such an embodiment, the reporter gene can be located either upstream or downstream of the regulated promoter. In another embodiment, the reporter gene is oriented in the same orientation as the regulated promoter. In such an embodiment, the reporter gene is preferably located upstream of the regulated promoter to avoid false reporter activity.

It will be apparent to one skilled in the art that any reporter genes that are functionally equivalent to any of the reporter genes as described, including any genes that are modified or mutated from any of the described reporter genes, are also within the scope of the present invention.

Rapid Cloning Element

The optional rapid cloning element comprises a bacterial plasmid replication origin and a bacterial selection marker. Any bacterial plasmid replication origin, such as but not limited to Ori, colEI, pSC101, pUC, or f1 phage ori, can be used. Any bacterial selection markers, such as but not limited to, chloramphenicol, ampicillin, tetracycline, or kanamycin, can be used in the present invention. The rapid cloning element functions as a selection bacterial plasmid to allow efficient cloning of the genomic DNA sequences flanking it into bacterial cells.

It will be apparent to one skilled in the art that any replication origins and bacterial selection marker genes that are functionally equivalent to any of the rapid cloning elements as described, including any genes that are modified or mutated from any of the described genes, are also within the scope of the present invention.

Additional Sequences

Depending on the particular gene search vector used, additional sequences may be necessary to be included in the vector. Such sequences and the manner of their inclusion in the vector are well within the knowledge of one skilled in the art and will be apparent to one skilled in the art when a particular vector is chosen. For example, the gene search vector may contain restriction sites to facilitate the manipulation of the vector. The gene search vector may also contain sequences that aid the integration of the vector into the host chromosome. When gene search vectors are introduced into cells by retroviral infection, sequences including long terminal repeats and packaging signals may also be included. The gene search vector can also comprise a sequence encoding an internal ribosome entry site (IRES) to permit independent translation of the selection marker gene (see, e.g., Jang et al., 1990, Genes Dev. 4:1560-72 and Ghattas et al., 1991, Mol. Cell. Biol. 11:5848-59). IRES is particularly useful in embodiments in which the selection marker gene is inserted in an intron or out of frame.

In embodiments for generation of a homozygous mutation at a targeted genomic locus, the gene search vector of the invention further comprises two sequence regions each comprising a nucleotide sequence which is substantially homologous to a target DNA sequence in the target genomic locus, one at each side of the DNA construct to be inserted into the genome, and a sequence region comprising a nucleotide sequence encoding a fluorescence marker for selection against random integration and located outside of the two homologous sequence regions. (See, e.g., Limin Li, U.S. Provisional Patent Application No. 60/325,450, filed on Sept. 27, 2001, which is incorporated herein by reference in its entirety.)

Methods of Introduction

The gene search vectors can be introduced into mammalian cells by any methods known in the art. The gene search vector compositions can be either transfected directly into mammalian cells by standard DNA transfection methods (such as microinjection, electroporation, and LIPOFECTAMINE) or by retroviral infection. In one embodiment, retroviral infection (Li et al., 1996, Cell 85:319) is used to introduce a gene search vector of the invention into the genome of cells. In another embodiment, a transposon (Ivics et al., 2000, Cell 91:501-510; Izsvak et al, 2000, J Mol Biol 302:93-102) method is used to introduce the gene search vector of the invention into the genome of cells. In still another embodiment, DNA transfection (Wigler et al., 1979, Cell 16:777-785) is used to introduce the gene search vector of the invention into the genome of cells.

Preferably, the transfection method is optimized for single copy integration of the gene search vector in cells (see, e.g., Li et al., 1996, Cell 85:319).

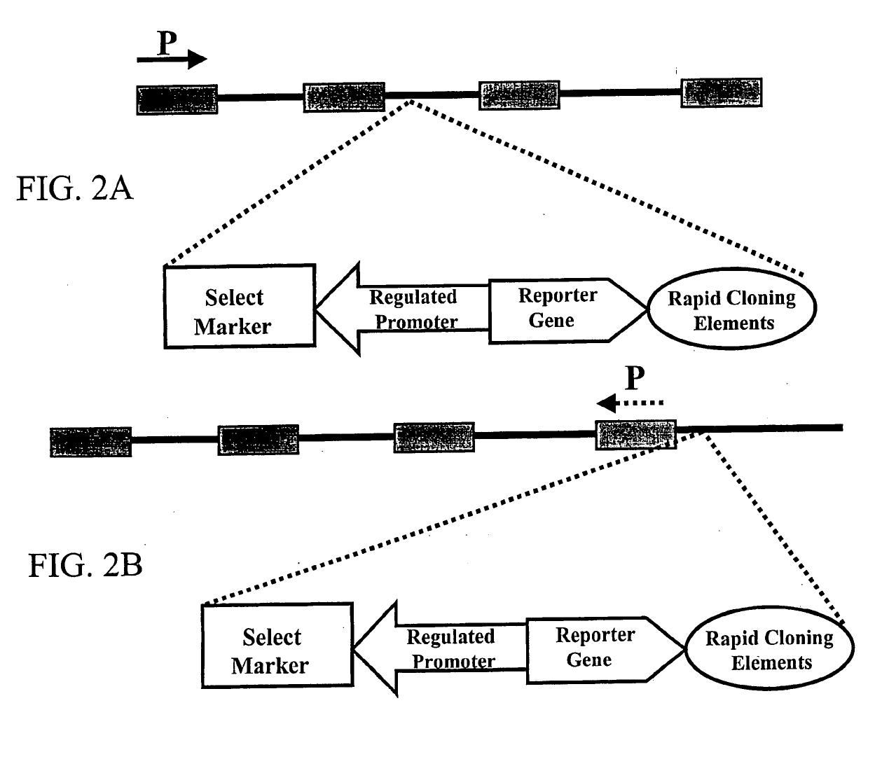

A gene search vector of the invention can be integrated into the genome of cells in two configurations (see FIGS. 2A and 2B). In one embodiment, the gene search vector integrates behind a chromosomal promoter. In this embodiment, the reporter gene is turned on by the chromosomal promoter. Integration of the gene search vector results in disruption of transcription at the allele (see FIG. 2A).

In another embodiment, the gene search vector integrates upstream of an inactive or active chromosomal promoter. In this embodiment, induction of the regulated promoter activates the inactive chromosomal promoter or amplifies the active chromosomal promoter (see FIG. 2B). This embodiment allows activation of chromosomal genes in cells to screen for any phenotypic changes associated to the activated gene.

Furthermore, multiple choices of selection markers and reporter genes used in the gene search vectors permits random inactivation or activation of multiple chromosomal genes in mammalian cells to screen any multigenic phenotypes, i.e., GFP reporter gene and Neomycin selection marker are used for inactivate or activate chromosomal gene X, and BFP reporter gene and Puromycin selection marker are used for inactivate or activate chromosomal gene Y in the same mammalian cell. In one embodiment, two or more gene search vectors are transfected into the same mammalian cell at the same time (cotransfection). In another embodiment, two or more gene search vectors are transfected into the same mammalian cell sequentially.

Methods of Generating Random Homozygous Genomic Mutations

The invention provides methods for generating random homozygous mutations at one or more genomic loci. Any gene search vector described in Section 5.1., supra, can be used for this purpose. In some preferred embodiments, a gene search vector is integrated behind a chromosomal promoter. In such embodiments, the gene search vector used for generating random homozygous mutations preferably comprises a reporter gene so that integration of the gene search vector downstream of a constitutive promoter, i.e., at an express chromosomal locus, can be identified. In one embodiment of the invention, the regulated promoter in the gene search vector is oriented in the same direction as the constitutive promoter of the chromosomal locus. In this embodiment, the reporter gene is located upstream of the regulated promoter in the gene search vector. In a preferred embodiment, the regulated promoter in the gene search vector is oriented in the opposite direction as the constitutive promoter of the chromosomal locus. In this embodiment, the reporter gene can be at either side of the regulated promoter. If the reporter gene is located upstream of the regulated promoter, it is preferably located upstream of the selection marker gene.

In some other preferred embodiments, the gene search vector is integrated upstream of an inactive or active chromosomal promoter. In such embodiments, the gene search vector will activate the inactive chromosomal promoter or amplify the active chromosomal promoter such that the integration of the gene search vector at an interesting site can be identified by a change in phenotype. In one embodiment of the invention, the regulated promoter in the gene search vector is oriented in the same direction as the constitutive promoter of the chromosomal locus. In a preferred embodiment, the regulated promoter in the gene search vector is oriented in the opposite direction as the constitutive promoter of the chromosomal locus.

In the methods of the invention, the gene search vector is introduced into a cell, e.g., a mammalian cell, to generate a single allelic genomic mutation at a genomic locus in the cell. Any method known in the art can be used for the random integration of the gene search vector into the genome of a cell. In one embodiment of the invention, random integration of a gene search vector is achieved by microinjection. In another embodiment, random integration of a gene search vector is achieved by electroporation or LIPOFECTAMINE. In still another embodiment, random integration of a gene search vector is achieved by retroviral infection. Preferably, the method used in the invention is optimized for single copy integration of the gene search vector in the cell.

In embodiments in which the gene search vector is integrated behind a chromosomal promoter, random integrations of the gene search vector in targeted cells, e.g., targeted mammalian cells, are preferably identified by the reporter gene, e.g., Puromycin resistance and GFP activity.

In embodiments in which the gene search vector is integrated upstream of a chromosomal promoter, random integrations of the gene search vector in the cells, e.g., mammalian cells, are preferably identified by screening of changes in phenotype, under conditions when the regulated promoter is fully activated.

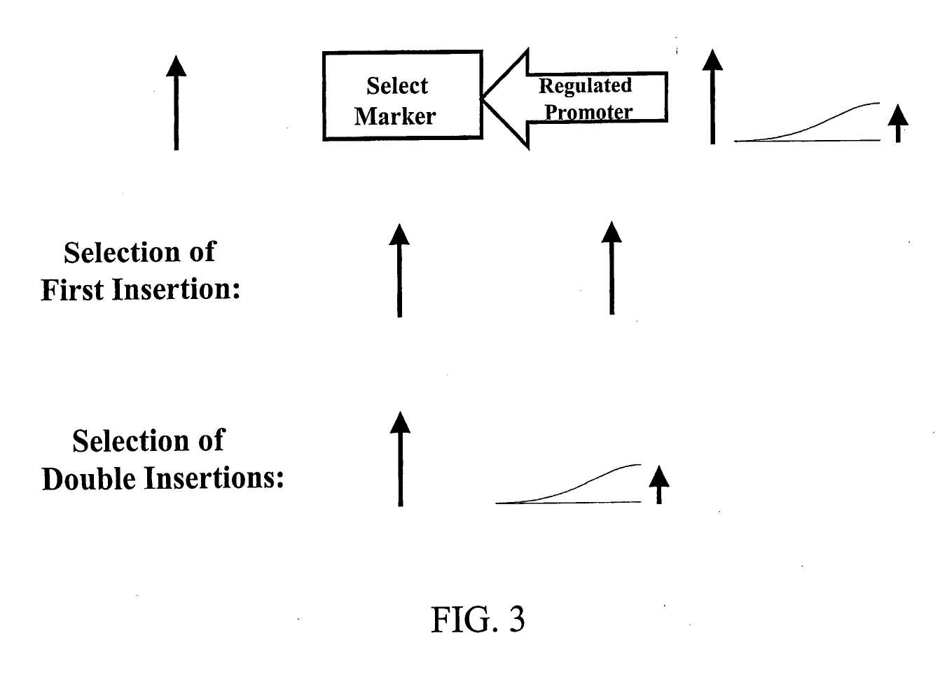

Due to the polyploid nature of cells, e.g., mammalian cells, complete gene inactivation generally requires the mutation of both alleles of the genomic locus. Two components (the selection marker and the regulated promoter) in the gene search vector are then used to convert a single allelic genomic mutation to mutations of both alleles of the genomic locus. In one embodiment, the conversion is accomplished by tuning the activity level of the regulated promoter to produce dosage and/or analogue dependent selection marker activity (FIG. 3). The activity level of the regulated promoter is partially activated to reduce the production of the selection marker, while the selection pressure for the selection marker is kept the same. In a preferred embodiment, the activity level of the regulated promoter is reduced to about 20% of the full activity level by reducing the concentration of the inducing agent. One skilled in the art will be able to determine the desired concentration of the inducing agent for this purpose. In one embodiment, the concentration of the inducing agent of the regulated promoter is adjusted such that below which no colonies showing activity of the positive selection marker are observed for a preselected period of culturing, whereas above which colonies showing activity of the positive selection marker are observed after such a period. Preferably, the preselected period is about 24 hours to 28 days. More preferably, the preselected period is about 24-72 hours.

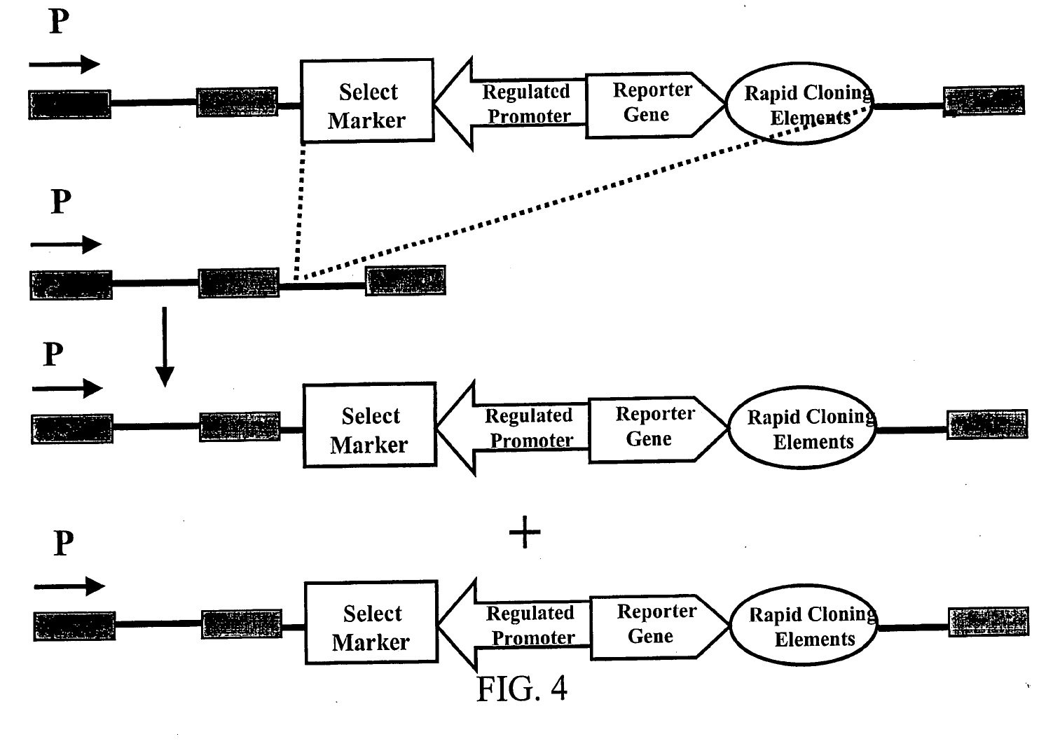

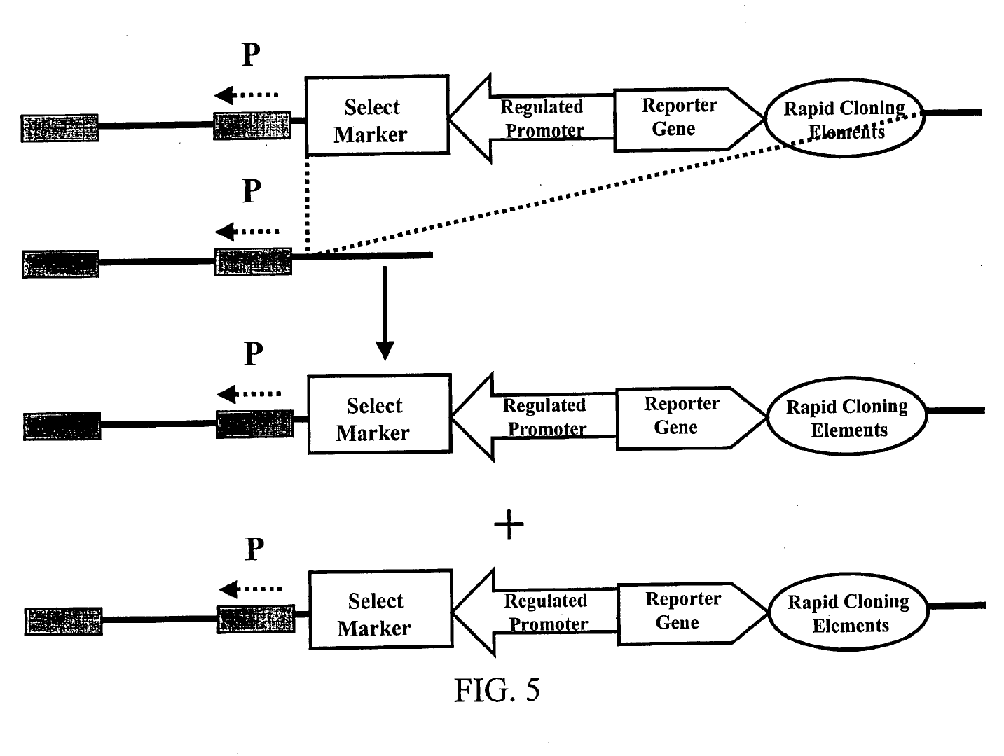

The selection pressure facilitates the selection of cells that have undergone conversion from a heterozygous genomic mutation to a homozygous genomic mutation, e.g., by homologous DNA recombination, at the same locus (FIGS. 4 and 5), which results in an increase of selection marker activity and a selective advantage over the heterozygous genomic mutation. In a preferred embodiment, the concentration of the inducing agent or an analogue of the inducing agent is set such that conversion from heterozygous genomic mutation to homozygous genomic mutation at the locus resulting in an 100% increase of selection marker activity. In another preferred embodiment, the concentration of the inducing agent or an analogue of the inducing agent is chosen such that under a given selection condition and within a given period of time cells that have undergone conversion from heterozygous genomic mutation to homozygous genomic mutation constitute at least a predetermined percentage of the resulted population of cells. Preferably, the concentration of the inducing agent or an analogue of the inducing agent is chosen such that under a given selection condition cells that have undergone conversion from heterozygous genomic mutation to homozygous genomic mutation constitute at least 1%, 5%, 10%, 20%, 50%, or 90% of the resultant population of cells within a given period of time. Preferably, the given period time is 24 hours, 72 hours, 7 days, 14 days, or 28 days.

In some other embodiments, selection pressure is adjusted, while the level activity of the regulated promoter is unchanged. In one embodiment, a drug resistance gene is used as the selection marker. In this embodiment, the selection pressure is increased by increasing the concentration of the corresponding drug. In preferred embodiment, the concentration of the drug is doubled.

In another preferred embodiment, a cell surface marker or a fluorescence marker is used as the selection marker. In this embodiment, two cell populations expressing different amounts of the selection marker can be observed. For example, in the embodiment in which a fluorescence marker is used as the selection marker, two cell populations, one displaying twice the fluorescence intensity of the other, can be observed. In this embodiment, the selection for cells which have undergone double allelic conversion can be achieved by selecting the population of cells that express the higher amount of the selection marker.

The genomic locus where the mutation takes place can be readily identified by cloning and sequencing the genomic sequences flanking the integration site and comparing them with a genomic sequence database. (See Section 5.7., infra)

Methods for Generating Targeted Homozygous Genomic Mutations

The invention provides methods for generating a targeted homozygous mutation at a genonic locus. In these methods, a single allelic mutation at a chosen genomic locus is first accomplished by inserting a copy of the DNA construct of the invention (as described in Section 5.1., supra) at the genomic locus. Any method known in the art that can be used for inserting in a chosen genomic locus a DNA construct can be used for this purpose.

Preferably, the single allelic mutation at the chosen genomic locus is generated by homologous recombination. In one embodiment, a positive-negative selection scheme that uses a negative selection marker for targeted insertion by homologous recombination is employed (see, e.g., U.S. Pat. Nos. 5,487,992; 5,627,059; 5,631,153; and 6,204,061, each of which is incorporated herein by reference in its entirety).

In another embodiment, a method utilizing a cell surface marker for selection against random integrations is employed (see, e.g., U.S. Pat. No. 6,284,541, which is incorporated herein by reference in its entirety). Selection for the absence of the negative selection marker is carried out by contacting the transfected cells with a binding molecule, e.g., a fluorescence dye tagged antibody, and identifying and isolating the cells using, e.g., a fluorescence activated cell sorter (FACS).

In a preferred embodiment, a method utilizes a selection scheme in which a selection marker gene that encodes a fluorescence protein, such as a green fluorescence protein, is employed for selection against random, non-homologous, insertions (see, e.g., Limin Li, U.S. Provisional Patent Application No. 60/325,450, filed on Sept. 27, 2001, which is incorporated herein by reference in its entirety). The method utilizes a gene targeting vector comprising four sequence regions: a first sequence region comprising a nucleotide sequence which is substantially homologous to a first DNA sequence in the targeted genomic locus; a second sequence region comprising a nucleotide sequence which is substantially homologous to a second DNA sequence in the targeted genomic locus; a third sequence region positioned between the first and second DNA sequence regions and comprising the DNA construct to be inserted, e.g., a DNA construct comprising a selection marker gene, a regulated promoter, a reporter gene for reporting integration of the DNA construct in the genome of the targeted cells, and an optional rapid cloning element; and a fourth sequence region comprising a nucleotide sequence located at 5′ to the first or 3′ to the second sequence region encoding a fluorescence marker for selection against random integration. Transfected cells that carry the insertion of the first through third sequence regions in the genome by homologous recombination can be selected by selecting for the presence of the positive selection marker activity and the absence of the activity of the selection marker or markers encoded in those outside regions, i.e., the fourth sequence region.

In a preferred embodiment, a drug resistance gene is used as the reporter gene for reporting integration of the DNA construct in the targeted genomic locus of the targeted cells. In this embodiment, the selection for cells carrying the insertion of the DNA construct can be achieved by culturing the transfected cells in the presence of the corresponding drug. In another preferred embodiment, a fluorescence marker is used as the reporter gene for reporting integration of the DNA construct in the genome of the targeted cells. In this embodiment, the selection for cells carrying the insertion of the DNA construct can be achieved by any fluorescence based cell sorting methods known in the art, e.g., by FACS. The selection against random, non-homologous, integration of the gene targeting vector can be carried out by detecting the fluorescence from the fluorescence marker encoded in the fourth sequence region using any fluorescence based cell sorting methods known in the art, e.g., by FACS. The method allows production of a cell population in which cells that carry the insertion of the DNA construct by homologous recombination constitute at least 10%, 30%, 50%, 70%, or 90% of the population by a fluorescence based cell sorting method.

The single allelic mutation is then converted into a double allelic mutation by methods described in Section 5.2., supra.

Methods for Generating Multiple Homozygous Genomic Mutations

The invention also provides methods for generating multiple homozygous genomic mutations. In the methods, multiple gene search vectors are used to generate homozygous mutations at a plurality of genomic loci. For this purpose, multiple choices of selection markers and reporter genes used in the gene search vectors permits random or targeted inactivation or activation of multiple chromosomal genes in cells, e.g., mammalian cells. Such methods are particularly useful to screen for any multigenic phenotypes.

Preferably, different combinations of regulated promoters and selection markers are used in different gene search vectors such that homozygous mutations generated by each gene search vector can be independently optimally carried out. In a specific embodiment, GFP reporter gene and Neomycin selection marker are used for inactivate or activate chromosomal gene X, and BFP reporter gene and Puromycin selection marker are used for inactivate or activate chromosomal gene Y in the same mammalian cell. In embodiments where more than one gene search vector has a rapid cloning element, it is preferred that each of the gene search vectors has a different rapid cloning element such that sequences from different genes can be independently cloned and characterized.

In one embodiment, the gene search vectors are transfected into the same mammalian cell at the same time (cotransfection).

In another embodiment, the gene search vectors are transfected into the same mammalian cell sequentially. In one embodiment, one or more gene search vectors are used to target and inactivate or activate one or more preselected genes to produce mutated cells. Random homozygous mutations are then generated using such mutated cells to produce cells demonstrating other new phenotypes. In a preferred embodiment, mutated cells produced by each gene search vector are retrieved and characterized. This embodiment is particularly useful in construction of libraries of mutated cells.

Library of Cells Carrying Homozygous Genomic Mutations

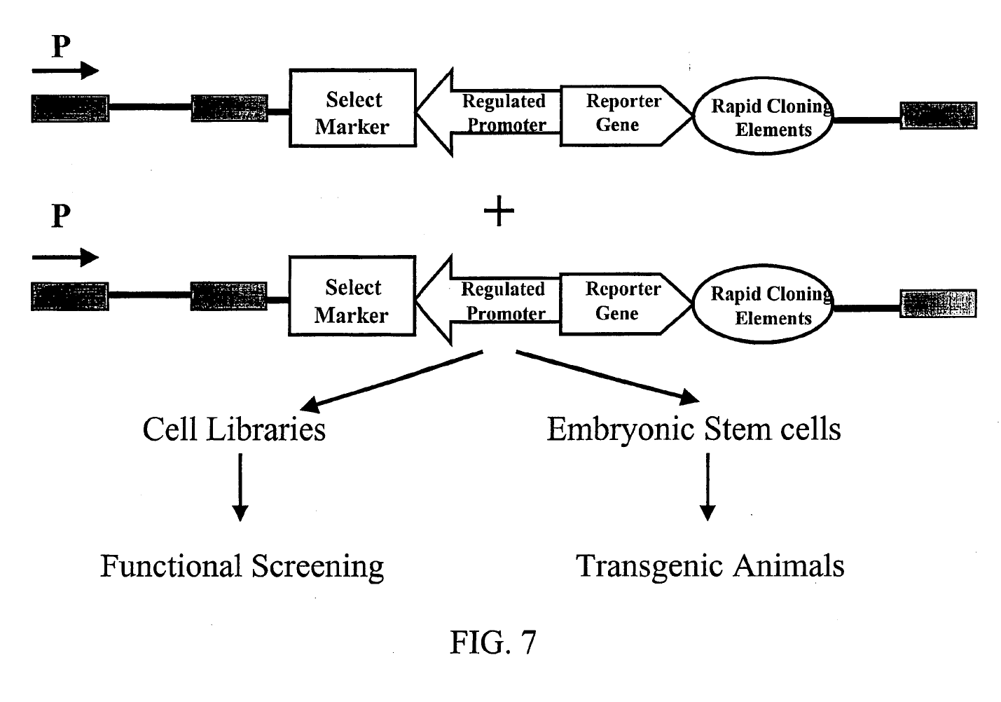

The invention also provides libraries of cells comprising a plurality of different cells, each of said plurality of different cells carrying a homozygous insertion of a gene search vector of the invention or portion thereof at one or more genomic loci (FIG. 7). Each of the different cells is generated by using any one of the methods of the invention. Preferably, the library of cells of the invention consists of at least 10, 100, 1,000, 10,000 different cells, each carrying different homozygous mutations in one or more genes. More preferably, the library of cells of the invention comprises for each gene in the genome of the cell at least one cell which carries a homozygous mutation in the gene.

Single Step Gene Knockout in Transgenic Animals and Transgenic Plants

The invention further provides a method for single step gene knockout in animals and plants (FIG. 7). Any one of the methods of the invention described supra can be used to introduce double insertion of a DNA construct into a genomic locus of a suitable type of animal cell, e.g., embryonic stem cells, or plant cell, e.g., tobacco leaf discs. The transformed animal cells or plant cells carrying the homozygous mutation are then used to generate the transgenic animal or plant without the tedious process of cross-breeding. Any standard method known in the art can be used to generate the transgenic animals or plants from the transformed animal or plant cells. In one embodiment, transgenic mice are generated by blastocyst injection of transformed mouse embryonic stem cells (see, e.g., Ramirez-Solis et al., in Methods in Enzymology, Vol. 225, pp. 855-878, 1993). In another embodiment, transgenic plants are regenerated from the transformed plant protoplast cells (see, e.g., Peters, Biotechnology: A Guide to Genetic Engineering, Dubuque, I A: Wm. C. Brown Publishers, 1993).

In a specific embodiment, the invention provides a transgenic animal, said transgenic animal carrying a homozygous insertion of a DNA construct at a genomic locus, said DNA construct comprising a selection marker linked to a regulated promoter, said method comprising (a) integrating said DNA construct at one allele of said genomic locus in one or more embryonic stem cells of said animal; (b) culturing said one or more embryonic stem cells under a concentration of an inducing agent to which said regulated promoter is responsive under a given selection condition such that embryonic stem cells having said homozygous mutation constitute at least a predetermined percentage of the resultant embryonic stem cell population within a given period of time; and (c) retrieving said embryonic stem cells having said homozygous mutation; and (d) generating said transgenic animal using said retrieved embryonic stem cells. In a preferred embodiment, the concentration of the inducing agent or an analogue of the inducing agent is set such that homozygous mutation at the locus resulting in an 100% increase of selection marker activity. In another preferred embodiment, the concentration of the inducing agent or an analogue of the inducing agent is chosen such that under a given selection condition and within a given period of time cells having homozygous mutation constitute at least a predetermined percentage of the resulted population of cells. Preferably, the concentration of the inducing agent or an analogue of the inducing agent is chosen such that under a given selection condition cells having homozygous mutation constitute at least 1%, 5%, 10%, 20%, 50%, or 90% of the resulted population of cells within a given period of time. Preferably, the given period time is 24 hours, 72 hours, 7 days, 14 days, or 28 days.

The invention therefore also provides transgenic organisms which carry homozygous insertion of a DNA construct of the invention at a genomic locus. The transgenic organism of the invention can carry any of the DNA constructs as described in Section 5.1., supra. A transgenic organism of the invention can be a transgenic animal, e.g., a trangenic mouse, or a transgenic plant. The invention further provides a library of transgenic organisms, comprising a plurality of different transgenic organisms of a same organism, each of said different transgenic organisms carries different homozygous mutations in one or more genes. Preferably, the library of transgenic organisms of the invention consists of at least 10, 100, 1,000, 10,000 different transgenic organisms, each carrying different homozygous mutations in one or more genes. More preferably, the library of transgenic organisms of the invention comprises for each gene in the genome of the transgenic organisms at least one transgenic organism which carries a homozygous mutation in the gene. In one embodiment, the library of transgenic organisms is a library of transgenic mice.

Methods for Characterization of Mutations

The invention provides methods for screening cells carrying insertion of one or more gene search vectors in both alleles of one or more genomic loci.

The invention provides methods for screening phenotypes. Any methods known in the art can be used for screening phenotypes. Different types of phenotypes may include changes in growth pattern and requirements, sensitivity or resistance to infectious agents or chemical substances, changes in the ability to differentiate or the nature of the differentiation, changes in morphology, changes in response to changes in the environment, e.g., physical changes or chemical changes, changes in response to genetic modifications, and the like. Double allelic knockout can be verified by standard methods known in the art, e.g., by real time PCR or Southern blotting.

Cells carrying homozygous mutations in one or more genes can be characterized by any methods known in the art. In one embodiment, the genomic region flanking the knockout construct DNA may be identified using PCR with the construct sequence as a primer for unidirectional PCR, or in conjunction with a degenerate primer, for bidirectional PCR. The sequence may then be used to probe a cDNA or chromosomal library for the locus, so that the region may be isolated and sequenced.

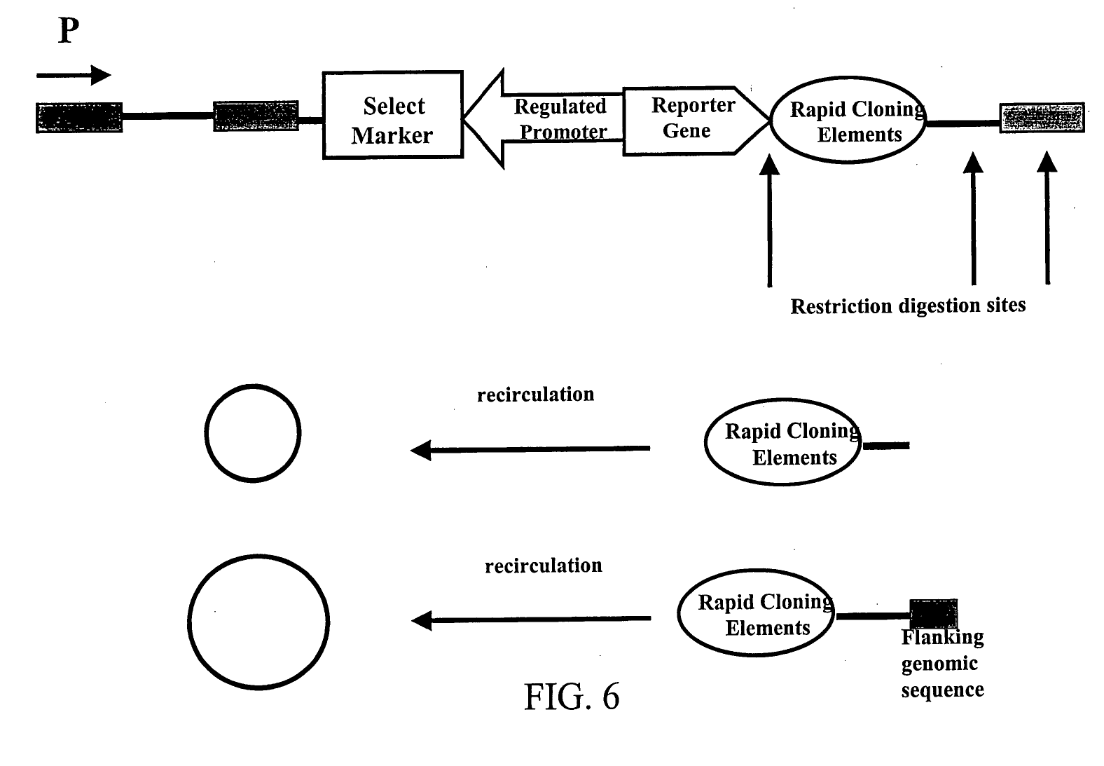

In a preferred embodiment, homozygous mutations are characterized by making use of the rapid cloning element (FIG. 6). In this embodiment, homozygous mutations are characterized by the following steps: first, the rapid cloning element and its flanking genomic DNA are digested by a single or two compatible restriction enzymes, then recirculized by DNA ligation, and transfected into cells of a bacterium. Alternatively, the Same splicing acceptor described above can be placed in either 5′ or 3′ of the rapid cloning elements, this allows the RNA transcript from flaking exons to fuse with rapid cloning elements. This RNA fusion transcript is then converted into double strand DNA by reverse transcriptase and DNA polymerase, recirculized by DNA ligase, and then transfected into bacterium. The plasmids isolated from transformed bacteria are used to determine the DNA sequence of the flanking exons by any DNA sequencing methods known in the art.

### EXAMPLE

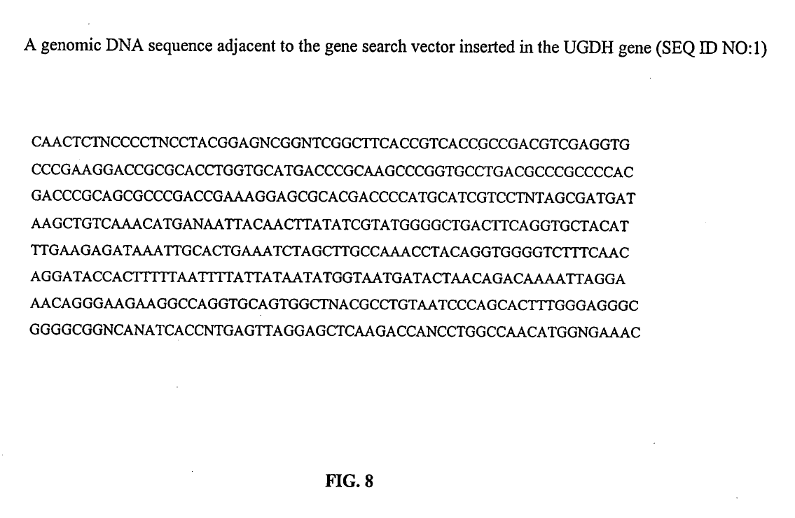

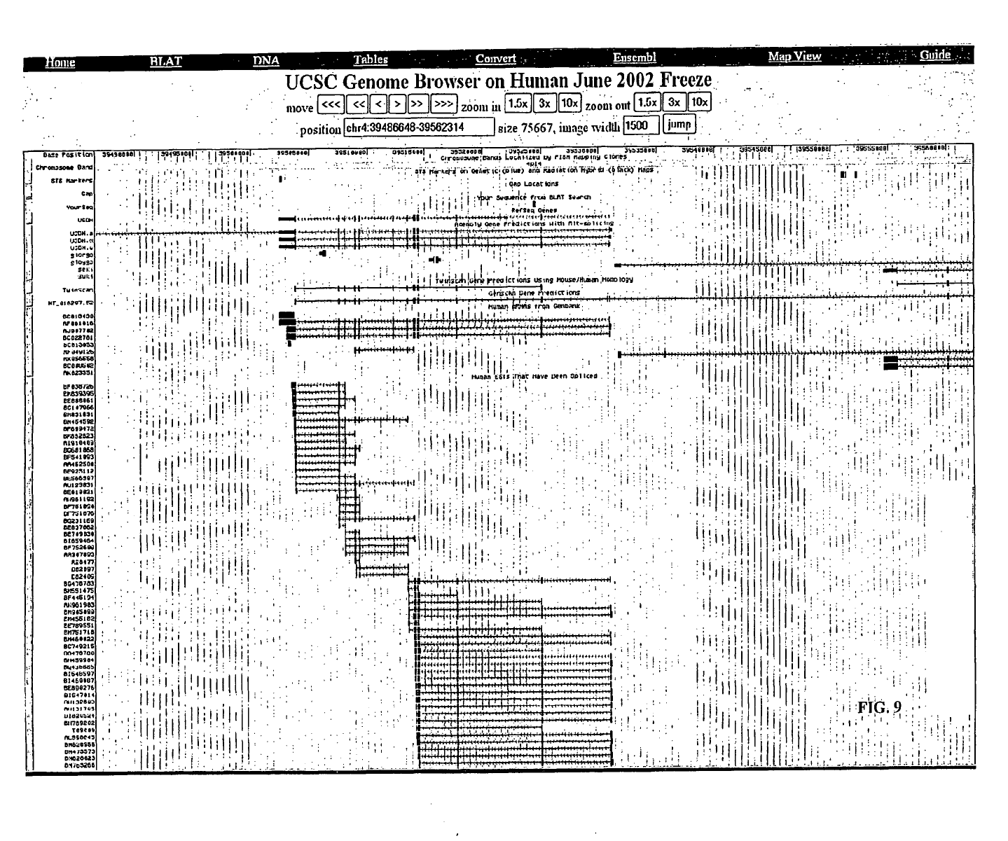

The following example describes the generation of homozygous mutations following random insertion of gene search vectors. The GFP-Neo gene search vector was used to generate retroviruses to infect a mouse neuroblastoma (N2a) cell line or a human breast cancer cell line (MDA-MB468). In the GFP-Neo gene search vector, the selection marker is a GFP and Neo fusion gene; the regulated promoter is a tetracycline regulated promoter; the reporter gene is BFP; and the rapid cloning element contains the bacterial selection marker chloramphenicol and a bacterial replication origin (see FIG. 1). 10 ug of gene search vector was transfected into phoenix helper cells by Lipofectamin (invitrogen). About 24 to 48 hours after the transfection, cell culture supernatant was collected and filtered with 0.2 um filter and used for infection of N2a or MDA-MB-468 cells. About 24 to 72 hours after the infection, the cells were trypsinized and resuspended, and sorted by FACS for BFP positive cells. Alternatively, about 24 to 72 hours after infection, the cells were selected using G418 (0.5 ug/ml) for neo expression or GFP expression in the presence of tetracycline induction. Clonal populations of cells were obtained either by FACS or by colony isolation, expanded into cell lines. More than 50 individual cell lines were obtained. Genomic DNA was then extracted from each cell line. The genomic DNA was digested either with Bam H1 or Hind III. The digested genomic DNA was phenol-chloroform extracted, precipitated and then ligated using ligase. The ligated genomic DNA was precipitated and used to transform E. Coli (Stable 4, Invitrogen) by electroporation. The transformed bacterial cultures were selected with Chloramphenicol (12.5 ug/ml), the resistant bacterial colonies were isolated and the plasmids were purified. Sequencing of the purified plasmids identified the genomic DNA sequences adjacent to the gene search vector. More than 40 genomic DNA sequences were determined. One of the infected human breast cancer cell lines contains an insertion of the gene search vector in the human UDP-glucose dehydrogenase gene (UGDH, NCBI Locus ID: 7358). In this cell line, the gene search vector was inserted between Exon 2 and Exon 3 of the human UDP-glucose dehydrogenase gene (see FIGS. 8 and 9).

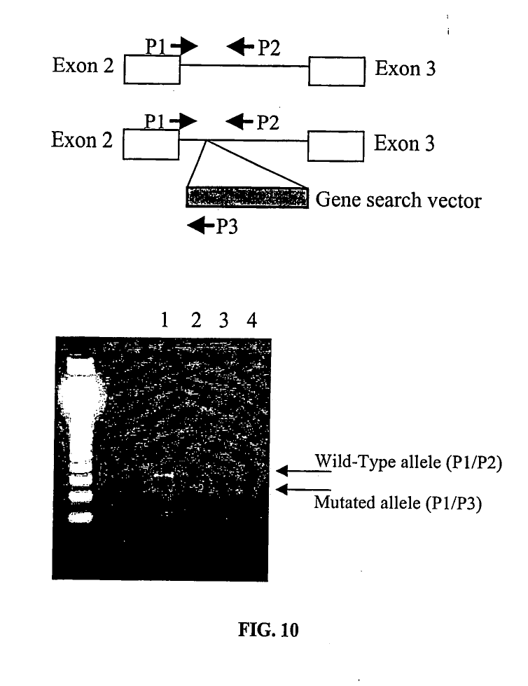

The cell line that contains the gene search vector insertion at UGDH was used for double conversion to generate homozygous mutation in the UGDH gene. 2×10⁵ cells were cultured with minimal induction by tetracycline (0.01 ug/ml). G418 resistant colonies were selected by culturing in 2 ug/ml of G418 for 4 weeks. 14 G418 resistant colonies were obtained, isolated and expanded into cell lines. Genomic DNA was extracted from each cell line, and used for genomic PCR analysis. Three PCR primers were designed to identify the wild-type allele and the mutated allele. P1 (5′ ctgttagtatcattaccatattat 3′, SEQ ID NO:2) and P2 (5′ tagaaaatgctaccatcaaatttg 3′, SEQ ID NO:3) PCR product (350 bp) identifies the wild-type allele, whereas P1 and P3 (5′cacctggtgcatgacccgcaagcccg3′, SEQ ID NO:4) identifies the mutated allele (see FIG. 10). Clone 4 contains only mutated alleles, demonstrating successful generation of homozygous mutation in the UGDH gene (see FIG. 10).

References Cited

All references cited herein are incorporated herein by reference in their entirety and for all purposes to the same extent as if each individual publication or patent or patent application was specifically and individually indicated to be incorporated by reference in its entirety for all purposes.

Many modifications and variations of the present invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

1. A method for generating a plurality of polyploid cells having a homozygous mutation at a genomic locus from one or more polyploid cells comprising a DNA construct integrated at one allele of said genomic locus, said DNA construct comprising a selection marker gene linked to a regulated promoter, said method comprising
culturing said one or more polyploid cells under a concentration of an inducing agent to which said regulated promoter is responsive under a given selection condition such that said plurality of polyploid cells having said homozygous mutation constitute at least a predetermined percentage of the resultant cell population within a given period of time, so as to generate a plurality of polyploid cells having said homozygous mutation.

2. The method of claim 1, further comprising retrieving said plurality of polyploid cells having said homozygous mutation.

3. The method of claim 1, wherein said one or more polyploid cells are generated by a method comprising, prior to said culturing, integrating said DNA construct into said allele of said genomic locus.

4. The method of claim 3, wherein said DNA construct further comprises a promoterless reporter gene, and wherein said method further comprises, before said culturing, a step of selecting cells by a method comprising selecting cells in which said reporter gene is transcribed.

5. The method of claim 1, wherein said DNA construct is integrated behind a chromosomal promoter at said genomic locus.

6. The method of claim 5, wherein said DNA construct inactivate the gene at said genomic locus.

7. The method of claim 1, wherein said DNA construct is integrated upstream of a chromosomal promoter at said genomic locus.

8. The method of claim 7, wherein said DNA construct activate the gene at said genomic locus.

9. The method of claim 2, wherein said DNA construct further comprises a rapid cloning element, and wherein said method further comprises a step of cloning genomic sequences flanking said DNA construct in a host, wherein said rapid cloning element comprises a replication origin.

10. The method of claim 9, wherein said rapid cloning element further comprises a bacterial selection marker.

11. The method of claim 9 or 10, wherein said step of cloning is carried out after said step of retrieving.

12. The method of claim 11, wherein said step of cloning comprises digesting the DNA of said plurality of cells with a restriction enzyme.

13. The method of claim 12, wherein said step of cloning further comprises recirculizing the restriction digestion fragments and transfecting the recirculized DNA molecules into bacterial cells.

14. The method of claim 13, wherein said method further comprises sequencing said restriction digestion fragments.

15. The method of any one of claims 1-10, wherein said regulated promoter is a regulated promoter responsive to tetracycline and wherein said inducing agent is tetracycline.

16. The method of claim 15, wherein said selection marker gene is a drug resistance gene.

17. The method of claim 16, wherein said drug resistance gene is a neomycin resistance gene.

18. The method of claim 15, wherein said selection marker gene is a gene encoding a cell surface marker.

19. The method of claim 18, wherein said cell surface marker is CD4.

20. The method of claim 15, wherein said selection marker gene is a gene encoding a fluorescence marker.

21. The method of claim 20, wherein said fluorescence marker is a green fluorescence protein.

22. A method for generating a homozygous mutation at a genomic locus in a type of polyploid cells, comprising
(a) integrating a DNA construct comprising a selection marker gene linked to a regulated promoter at one allele of said genomic locus in one or more cells of said type of polyploid cells; and
(b) culturing said one or more cells under a concentration of an inducing agent to which said regulated promoter is responsive under a given selection condition such that cells having said homozygous mutation at said genomic locus constitute at least a predetermined percentage of the resultant cell population within a given period of time, so as to generate said homozygous mutation in said type of polyploid cells.

23. The method of claim 22, wherein said DNA construct further comprises an rapid cloning element, said rapid cloning element comprising an replication origin, and wherein said method further comprises a step of cloning genomic sequences flanking said DNA construct in a host.

24. The method of claim 23, wherein said rapid cloning element further comprises a bacterial selection marker.

25. The method of claim 23 or 24, wherein said step of cloning is carried out after said step of retrieving.

26. The method of claim 25, wherein said step of cloning comprises digesting the DNA of said plurality of cells with a restriction enzyme.

27. The method of claim 26, wherein said step of cloning further comprises recirculizing the restriction digestion fragments and transfecting the recirculized DNA molecules into bacterial cells.

28. The method of claim 27, wherein said method further comprises sequencing said restriction digestion fragments.

29. A population of polyploid cells, comprising cells that carry a homozygous insertion of a DNA construct at a genomic locus, wherein said DNA construct comprises a selection marker linked to a regulated promoter.

30. The population of polyploid cells of claim 29, wherein said polyploid cells are mammalian cells.

31. The population of polyploid cells of claim 30, wherein said mammalian cells are embryonic stem cells.

32. The population of polyploid cells of claim 30 or 31, wherein said mammalian cells are cells of a mouse.

33. The population of polyploid cells of claim 29, wherein said polyploid cells are plant cells.

34. A library of polyploid cells, comprising a plurality of different polyploid cells, wherein each of said plurality of different polyploid cells carries a different homozygous mutation in one or more genes.

35. The library of polyploid cells of claim 34, wherein said library consists of at least 10 different polyploid cells.

36. The library of polyploid cells of claim 35, wherein said library consists of at least 100 different polyploid cells.

37. The library of polyploid cells of claim 36, wherein said library consists of at least 1,000 different polyploid cells.

38. The library of polyploid cells of claim 37, wherein said library consists of at least 10,000 different polyploid cells.

39. The library of polyploid cells of claim 38, wherein said library comprises for each gene in the genome of said polyploid cells at least one polyploid cell wherein said gene contains a homozygous mutation.

40. A transgenic organism, wherein said transgenic organism carries a homozygous insertion of a DNA construct at a genomic locus, wherein said DNA construct comprises a selection marker linked to a regulated promoter.

41. The transgenic organism of claim 40, wherein said transgenic organism is a transgenic animal.

42. The transgenic animal of claim 41, wherein said transgenic animal is a transgenic mouse.

43. The transgenic organism of claim 40, wherein said transgenic organism is a transgenic plant.

44. A library of transgenic organisms, comprising a plurality of different transgenic organisms, wherein each of said different transgenic organisms carries a different homozygous mutations in one or more genes.

45. The library of transgenic organisms of claim 44, wherein said library consists of at least different transgenic organisms.

46. The library of transgenic organisms of claim 45, wherein said library consists of at least 100 different transgenic organisms.

47. The library of transgenic organisms of claim 46, wherein said library consists of at least 1,000 different transgenic organisms.

48. The library of transgenic organisms of claim 47, wherein said library consists of at least 10,000 different transgenic organisms.

49. The library of transgenic organisms of claim 48, wherein said library comprises for each gene in the genome of said organism at least one transgenic organism wherein said gene contains a homozygous mutation.

50. The library of transgenic organisms of any one of claims 44-49, wherein said transgenic organisms are transgenic animals.

51. The library of transgenic animals of claim 50, wherein said transgenic animals are transgenic mice.

52. The library of transgenic organisms of any one of claims 44-49, wherein said transgenic organisms are transgenic plants.

53. A method for generating a plurality of polyploid cells having homozygous mutations at a plurality of genomic loci from one or more polyploid cells comprising a plurality of DNA constructs each integrated at one allele of one of said plurality of genomic loci, each DNA construct in said plurality of DNA constructs comprising a selection marker gene linked to one of a plurality of regulated promoters, said method comprising
culturing said one or more polyploid cells under a concentration of each of a plurality of inducing agents to which one of said regulated promoters is responsive under a given selection condition such that said plurality of polyploid cells having said plurality of homozygous mutations constitute at least a predetermined percentage of the resultant cell population within a given period of time, so as to generate said plurality of polyploid cells having said homozygous mutations.

54. A method for generating homozygous mutations at a plurality of genomic loci in a type of polyploid cells, comprising
(a) integrating a plurality of DNA constructs each at one allele of one of said plurality of genomic loci in one or more polyploid cells of said type of polyploid cells, wherein each of said plurality of DNA construct comprises a selection marker gene linked to a regulated promoter; and
(b) culturing said one or more polyploid cells under a concentration of each of a plurality of inducing agents to which one of said regulated promoters is responsive under a given selection condition such that said plurality of polyploid cells having said plurality of homozygous mutations constitute at least a predetermined percentage of the resultant cell population within a given period of time, so as to generate said homozygous mutations at said plurality of genomic loci in said type of polyploid cells.

55. A method for generating a transgenic animal, said transgenic animal carrying a homozygous insertion of a DNA construct at a genomic locus, said DNA construct comprising a selection marker gene linked to a regulated promoter, said method comprising
(a) integrating said DNA construct at one allele of said genomic locus in one or more embryonic stem cells of said animal;
(b) culturing said one or more embryonic stem cells under a concentration of an inducing agent to which said regulated promoter is responsive under a given selection condition such that embryonic stem cells having said homozygous mutation constitute at least a predetermined percentage of the resultant embryonic stem cell population within a given period of time; and
(c) retrieving said embryonic stem cells having said homozygous mutation; and
(d) generating said transgenic animal from said retrieved embryonic stem cells, so as to generate said transgenic animal.

56. The method of any one of claims 1-10, 22-24, and 53-55, wherein said predetermined percentage is at least 1%.

57. The method of claim 56, wherein said predetermined percentage is at least 5%.

58. The method of claim 57, wherein said predetermined percentage is at least 10%.

59. The method of claim 58, wherein said predetermined percentage is at least 20%.

60. The method of claim 59, wherein said predetermined percentage is at least 50%.

61. The method of claim 60, wherein said predetermined percentage is at least 90%.

62. The method of any one of claims 1-10, 22-24, and 53-55, wherein said given period is 24 hours.

63. The method of claim 62, wherein said given period is 72 hours.

64. The method of claim 63, wherein said given period is 7 days.

65. The method of claim 64, wherein said given period is 14 days.

66. The method of claim 65, wherein said given period is 28 days.

67. A method for generating homozygous mutations at a genomic locus in a type of polyploid cells, comprising
(a) integrating a DNA construct comprising a selection marker gene at one allele of said genomic locus in one or more cells of said type of polyploid cells;
(b) expanding said one or more cells; and
(c) selecting cells such that cells having said homozygous mutation at said genomic locus constitute at least a predetermined percentage of the resultant cell population within a given period of time;
so as to generate said homozygous mutation in said type of polyploid cells.
